(19) United States
(12) Patent Application Publication
(10) Pub. No.: US 2023/0172758 A1
(43) Pub. Date: Jun. 8, 2023

(54) AUGMENTED WOUND DRESSING SYSTEMS AND METHODS FOR USING SAME AND SYSTEMS, DEVICES, AND METHODS FOR MONITORING THE HEALING PROCESS OF WOUNDS

(71) Applicant: Gregory O'KEEFFE, Boston, MA (US)

(72) Inventor: Gregory O'KEEFFE, Boston, MA (US)

(21) Appl. No.: 17/927,678

(22) PCT Filed: May 26, 2021
(86) PCT No.: PCT/US21/34326

§ 371 Date: Nov. 23, 2022

Related U.S. Application Data
(60) Provisional application No. 62/704,739, filed on May 26, 2020.

(51) Int. Cl. A61F 13/00

(52) U.S. Cl. CPC A61F 13/00055; A61F 2013/00953; A61F 2013/0097; A61F 2013/00944; A61F 2013/00948; A61F 2013/00957

(57) ABSTRACT

Augmented wound dressing systems may include traditional wound dressing components and one or more sensor devices such as accelerometers, humidity monitors, temperature monitors, electrocardiography leads, and/or oximetry sensors. Measurements from the sensor devices may be used to monitor one or more aspects of patient and/or wound health. In addition, or alternatively, one or more images of a wound that may be taken over time may be analyzed and/or compared with one another to determine one or more characteristics of the wound in order to, for example, assess wound health/healing and determine whether an intervention is necessary to assist with wound healing.

### RELATED APPLICATION

This patent application is a NON-PROVISIONAL patent application claiming priority to U.S. Provisional Patent Application No. 62/704,739, filed on 26 May 2020 and entitled “AUGMENTED WOUND DRESSING SYSTEMS AND METHODS FOR USING SAME AND SYSTEMS, DEVICES, AND METHODS FOR MONITORING THE HEALING PROCESS OF WOUNDS,” which is incorporated in its entirety herein.

### FIELD OF INVENTION

The present invention pertains to wound dressings and the monitoring of the healing process of wounds. In particular, the present invention relates to augmented wound dressing systems and methods for using same and systems, devices, and methods for monitoring the healing process of wounds.

### BACKGROUND

Following surgery, or bodily insult resulting in wound, a wound dressing is typically applied to keep the wound dry and limit exposure of the wound to potential pathogens that may enter the wound and cause an infection. Traditionally, these wound dressings are limited to an adhesive material configured to attach the wound dressing to the patient's skin and an absorbent material configured to wick fluids away from the wound and do not offer any ability to monitor the health of the patient or the wound.

Without direct observation, it is difficult to monitor wound healing, which makes remote monitoring of wound health is difficult. As such, it is currently difficult for clinicians to remotely assess whether a status for the healing of a wound is in need of an intervention to prevent, for example, infection, further injury, and/or scarring, which inhibits a clinician's ability to care for his/her patient. In addition, patients typically don't seek medical attention for a wound until a substantial infection has developed, which often triggers more invasive care (e.g., intravenous antibiotics or surgery) that is undesirable for a number of reasons, patient discomfort along with increased costs and hospital readmission rates.

### SUMMARY

Augmented wound dressings and augmented wound dressings systems may include traditional wound dressing components and one or more measurement, sensor, or monitor devices such as accelerometers, humidity monitors, temperature monitors, electrocardiography leads, and/or oximetry sensors. Measurements from the sensor devices may be used to monitor one or more aspects of patient and/or wound health including, but not limited to, patient mobility, respiratory function, cardiac function, blood oxygenation, body temperature, possibility of wound infection and/or wound healing rate.

The augmented wound dressings disclosed herein may include a wound dressing pad configured to cover a patient's wound and one or more monitor(s) configured to measure one or more aspects of the patient's health. Often times, the wound dressing may include a wound pad adhesive configured to adhere the wound dressing to the patient. In embodiments where two or more monitors are used, the two or more monitors may cooperate together to monitor an aspect of the patient's health.

In some embodiments, the wound dressing pad may be configured to absorb moisture and/or protect the wound. At times, the wound dressing pad is transparent or semi-transparent. In some embodiments, the wound dressing pad is configured to be elastic in order to, for example, apply a compressive force to a wound and/or move/flex with a patient when the patient moves. At times, the monitor is affixed to the wound dressing pad or the wound dressing adhesive.

In some embodiments, the wound dressing may have a plurality of monitors. Exemplary monitors include, but are not limited to, an accelerometer, a humidity monitor, a temperature monitor, one or more electrocardiography leads, a blood oxygen saturation sensor, a tissue oxygen saturation sensor, or a blood sugar sensor. At times, the monitor may include a transmitter configured to communicate a measurement of an aspect of the patient's health to an external computing device.

In addition, or in the alternative, one or more images of a wound that may be taken over time may be analyzed and/or compared with one another to determine one or more characteristics of the wound in order to, for example, assess wound health/healing and determine whether an intervention is necessary to assist with wound healing.

Exemplary methods disclosed herein may include receiving identifying information for a wound on a patient, receiving an image of the wound, the image being associated with color balancing information, determining a characteristic of the wound using the image and the coloring balancing information, and communicating the characteristic to a display device for display to a user. The determined characteristic may be, for example, a length of the wound, a width of the wound, a size, or a type of wound, a height of the wound, a texture of the wound and/or surrounding tissue, and a temperature of the wound and/or surrounding tissue.

Optionally, the method may further include generating a color-corrected image of the wound and communicating the color-corrected image of the wound to the display device for display to the user. When a color-corrected image of the wound is generated, the determining of the characteristic of the wound may include analysis of the color-corrected image of the wound.

In some embodiments, a plurality of images of the wound may be received over time and compared with one another to determine, for example, how healing of the wound is progressing over time. This may include receiving a second (or third, or fourth) image of the wound, the second image being associated with color balancing information, generating a second color-corrected image of the wound, comparing the first color-corrected image of the wound and the second color-corrected image of the wound, determining a second characteristic of the wound using a result of the comparing of the first color-corrected image of the wound and the second color-corrected image of the wound, and communicating the characteristic to a display device for display to a user.

### BRIEF DESCRIPTION OF THE DRAWINGS

The present invention is illustrated by way of example, and not limitation, in the figures of the accompanying drawings in which:

FIG. 1 is a block diagram of an exemplary augmented wound dressing system, consistent with some embodiments of the present invention;

FIG. 2 is a flowchart illustrating an exemplary process for determining a wound's status, consistent with some embodiments of the present invention;

FIG. 3A is an illustration of a wound in the form of a freshly made surgical incision, consistent with some embodiments of the present invention;

FIG. 3B is an illustration of the wound a few days following when the incision of FIG. 3A was made that is healing as expected, consistent with some embodiments of the present invention;

FIG. 3C is an illustration of the wound a few days following when the incision of FIG. 3A was made that is not healing as expected and/or is infected, consistent with some embodiments of the present invention; and

FIG. 4 is a block diagram of an exemplary processor-based system that may store data and/or execute instructions for the processes disclosed herein, in accordance with some embodiments of the present invention.

Throughout the drawings, the same reference numerals and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### WRITTEN DESCRIPTION

The augmented wound dressings and augmented wound dressing systems disclosed herein may operate to cover or protect a wound and may house and/or cooperate with one or more monitoring devices configured to monitor an aspect of the patient's health and/or movement. The augmented wound dressings and augmented wound dressing systems disclosed herein may be configured to cover, protect, and/or absorb moisture from a wound. Exemplary wounds include any break and/or damage to a patient's skin and include, but are not limited to, surgical incisions, cuts, tears, burns, abrasions, rashes, skin grafts, and abscesses. The augmented wound dressings and augmented wound dressing systems disclosed herein may be configured in any number of forms, but not limited to, wound dressings of different shapes (e.g., square, rectangle, circle). In some embodiments, augmented wound dressing systems disclosed herein may be a wound dressing with integrated butterfly sutures and/or a transparent, or semi-transparent window to allow for observation and/or photographs of the wound.

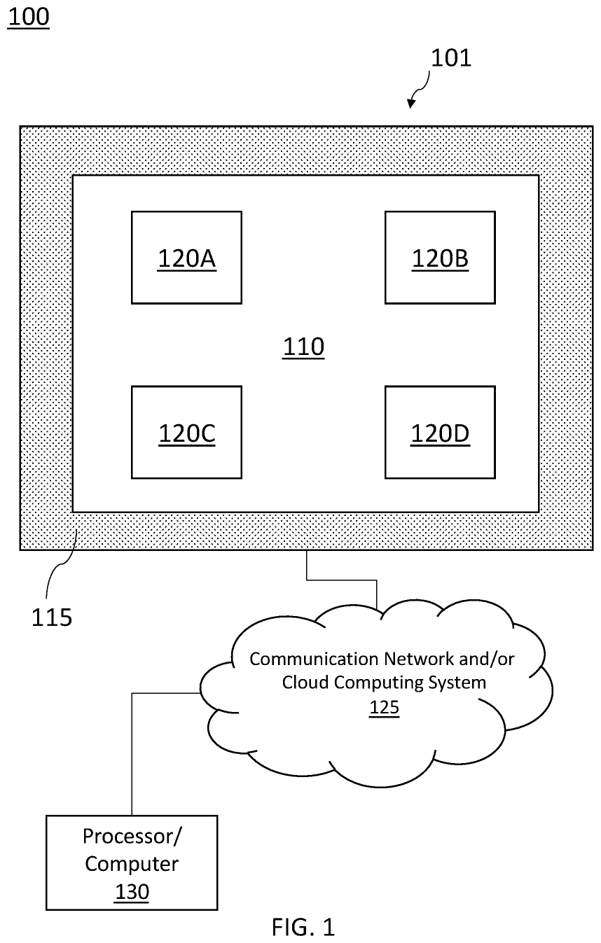

FIG. 1 is a block diagram of an exemplary augmented wound dressing system 100 that includes an augmented wound dressing 101, which includes a wound dressing pad 110 along with an optional wound pad adhesive 115, a communication network and/or a cloud computing system 125, and a processor and/or computer 130. Wound dressing pad 110 may be a pad of any material that may cover a wound. In some embodiments, wound dressing pad 110 is absorbent but this need not always be the cases. In some instances, wound dressing pad 110 is transparent so that an underlying wound may be viewed or observed for signs of, for example, healing and/or infection. Wound pad adhesive 115 may be any appropriate adhesive or glue. Augmented wound dressing 101 may be applied to a patient's skin to cover a wound using any appropriate means (e.g., tape, a bandage, etc.) in addition, or alternatively, to wound pad adhesive 115.

In some embodiments, a plurality of monitors 120 may be included in an augmented wound dressing 101. For example, FIG. 1 shows an exemplary augmented wound dressing 101 with four monitors 120: a first monitor 120A, a second monitor 120B, a third monitor 120C, and a fourth monitor 120D. One or more monitors 120 of an augmented wound dressing 101 may communicate with a processor like processor/computer 130 via, for example, a transmitter and/or transceiver that is resident in the respective monitor 120 and/or is a separate component of augmented wound dressing 101. In many cases, the communication between a monitor 120 and the processor will be via communication network 125, which may be configured to, for example, facilitate communication between one or more monitors 120 and processor/computer 130 and/or a cloud computing network resident within and/or communicatively coupled to communication network 124. Exemplary communication networks 125 include, but are not limited to, the Internet, a Wi-Fi network, and/or a mesh network. Processor/computer 130 may be configured to, for example, receive information from one or more monitors and provide that information to a user and/or clinician who is monitoring a patient's health. Exemplary processor/computers 130 include, but are not limited to personal computers, smart phones, and tablet computers.

The one or more monitors 120 of an augmented wound dressing 101 may be configured to monitor one or more aspects of a patient's health and/or a patient's wound. For example, a monitor 120 may be an accelerometer, a humidity sensor, one or more electrocardiography leads, a temperature sensor, a blood oxygen saturation sensor, a tissue oxygen saturation sensor, and/or a blood sugar sensor. In some embodiments, augmented wound dressing 101 may be configured so that a monitor 120 is in contact with a patient's skin proximate to the wound as may the case for monitors 120 that are, for example, electrocardiography leads, a temperature sensor, a humidity sensor, a temperature sensor, a blood oxygen saturation sensor, a tissue oxygen saturation sensor, and/or a blood sugar sensor. Additionally, or alternatively, a monitor 120 may be placed on the outside of augmented wound dressing 101 as may the case when monitor 120 is, for example, an accelerometer.

When monitor 120 is an accelerometer, readings, or measurements, it takes may be analyzed to determine, for example, a respiratory rate of the patient (when the augmented wound dressing 101 is positioned on the thorax of the patient), a level of physical activity for a patient, a degree of vigor for that activity, and/or an orientation of the patient (e.g., how long the patient is reclining, sitting, or standing). Additionally, or alternatively, accelerometer measurements taken over a period of time may be analyzed to determine changes to a level of physical activity for the patient whereby, for example, a steady increase in physical activity during recovery associated with a wound that is covered by augmented wound dressing 101 may indicate an improvement in the patient's condition and/or healing of the wound and/or an underlying condition that led to the wound. Likewise, a plateau or decrease of physical activity as indicated by the accelerometer monitor 120 may indicate that the patient is not improving, the wound is not healing, and/or that the patient needs an intervention (e.g., a telehealth or in-person visit with, for example, a medical care giver, social worker, or doctor) to determine if he or she needs further assistance (e.g., a change in medication or a visit to a hospital).

When monitor 120 is a tissue or blood oxygen saturation monitor, readings, or measurements, it takes may be analyzed to determine blood oxygenation levels and/or local tissue oxygen saturation, which may be an indicator of wound health and/or likelihood of healing or becoming infected because, in many cases, poor tissue oxygenation may increases a likelihood of wound infection and an indication of low tissue oxygenation may be interpreted as an alert to a clinician that a wound is, or may soon become, infected.

In some embodiments, a wound dressing may be transparent, include a transparent window, and/or may be temporarily removed from a wound (e.g., peeled away) so that an image, or photograph, of underlying wound may be taken. At times, these wound dressings may include an identifier that, when captured in the image, may be used to identify the wound and/or a patient wearing the wound dressing. The identifier may be, for example, an alpha-numeric code and/or an optical code (e.g., bar or QR code).

The image/photograph may be color-corrected or white balanced so that the colors of the image are accurately represented (and, in some cases, repeatable) regardless of lighting conditions. The presence of an identifier in an image of a wound may enable the aggregation and/or sorting of images of the patient's wound which may be taken over time without, for example, the need for any personally identifiable information about the patient to be a part of the analysis process. In some embodiments, augmented wound dressing 101 may include and/or be configured to have a sizing and/or color balancing object and/or graphic that may be used to, for example, establish a scale or size of a wound appearing in an image and/or a color, or range of colors, of a wound appearing in an image.

In some embodiments, wound dressing pad 110, wound dressing adhesive 115, and/or portions thereof may be configured to have elastic qualities so that, for example, a wound may be held together via a compressive force exerted by wound dressing system's 100 elastic properties. Additionally, or alternatively, elastic qualities of augmented wound dressing 101 may enable augmented wound dressing 101 to move with a wearer during, for example, breathing, coughing, or walking without pulling on, or away from, the wearer's skin. Wound dressing system's 100 elastic properties may be achieved by, for example, inclusion of elastic materials (e.g., rubber, latex, SPANDEX™, etc.) in, for example, wound dressing pad 110 and/or wound pad adhesive 115. Additionally, or alternatively, wound dressing system's 100 elastic properties may be achieved by a manner to construction of wound dressing pad 110 and/or wound pad adhesive 115 so that wound dressing pad 110 and/or wound pad adhesive 115 is configured to expand and contract via, for example, a woven or other pattern of manufacture.

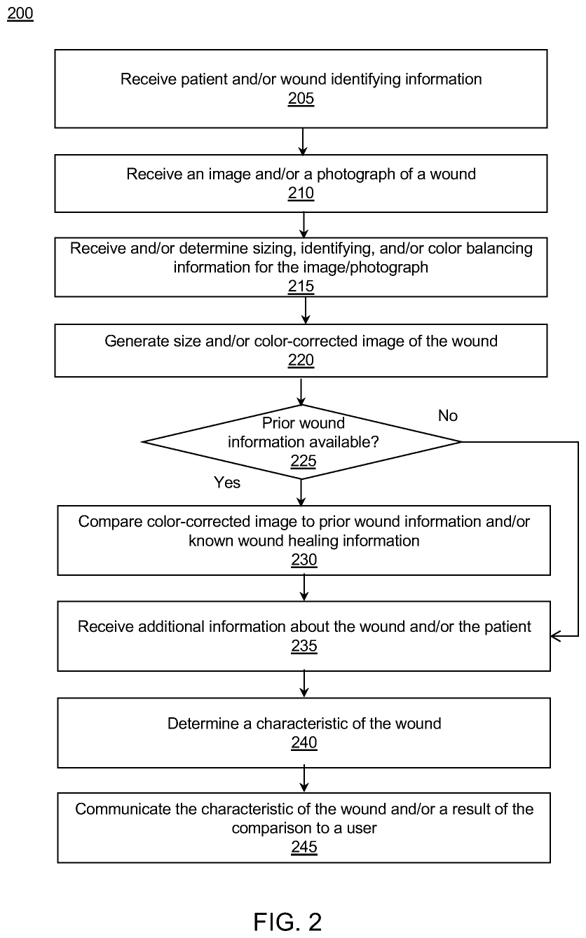

FIG. 2 provides a flowchart of an exemplary process 200 for the determination of a wound's status using images and/or photographs of the wound taken over time. FIG. 2 may be executed by a computer or processor configured to execute process 200 such as processor/computer 130 and/or processor-based system 400. In some instances, process 200 may be executed using artificial intelligence.

In step 205, patient and/or wound identifying information may be received by a processor from the patient via, for example, a personal electronic device (e.g., smart phone for tablet computer) operated by the patient. The patient-identifying information may be an identifier associated with a patient account that is provided via, for example, a sign-in process executed on the personal electronic device.

In step 210, an image and/or a photograph of a wound may be received by the processor. In some cases, the image and/or photograph may be communicated to the processor from the patient's personal electronic device and/or a device used in a medical facility like a hospital like a tablet computer or smart phone. In some instances, the image and/or photograph of the wound may be two-dimensional, three-dimensional, and/or a thermal image. In some instances, the image and/or photograph of the wound may be taken through a transparent wound dressing such as augmented wound dressing 101 and, in other instances, the image and/or photograph of the wound may be taken following removal of a wound dressing from the wound. At times, a wound dressing for the wound may include an identifier (e.g., alpha numeric and/or optical code) that identifies the wound dressing, the wound, and/or the patient.

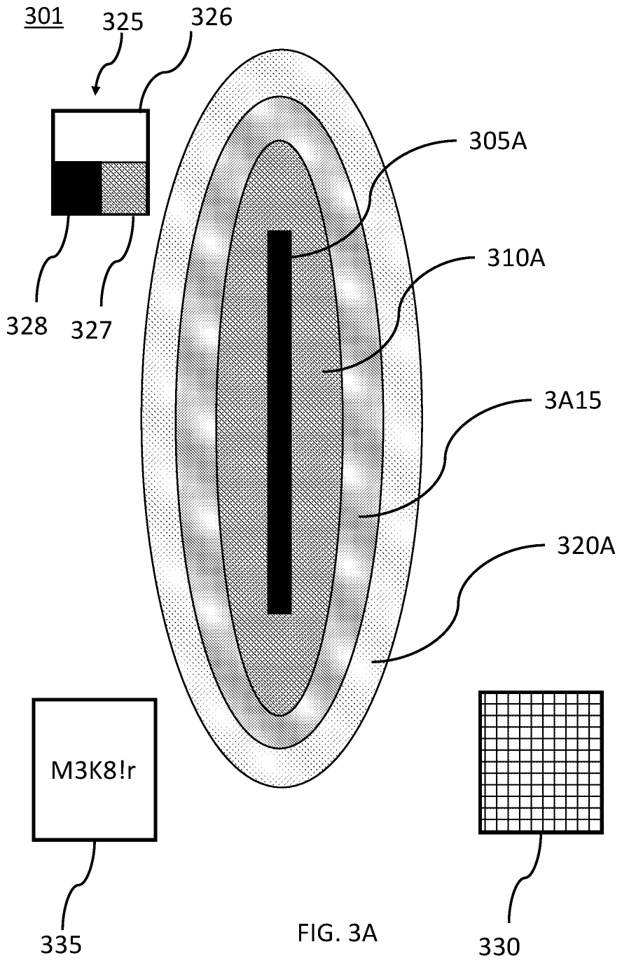

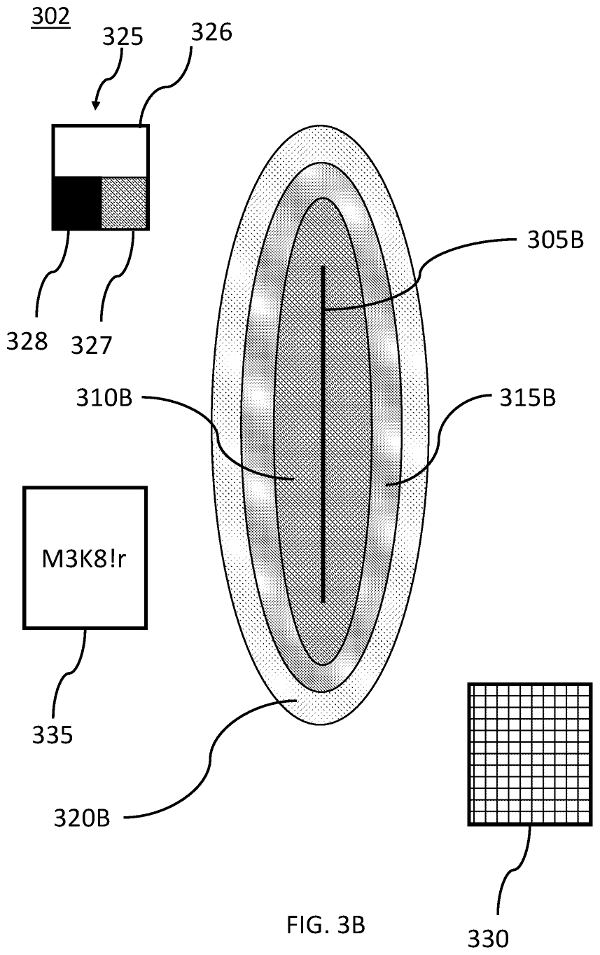

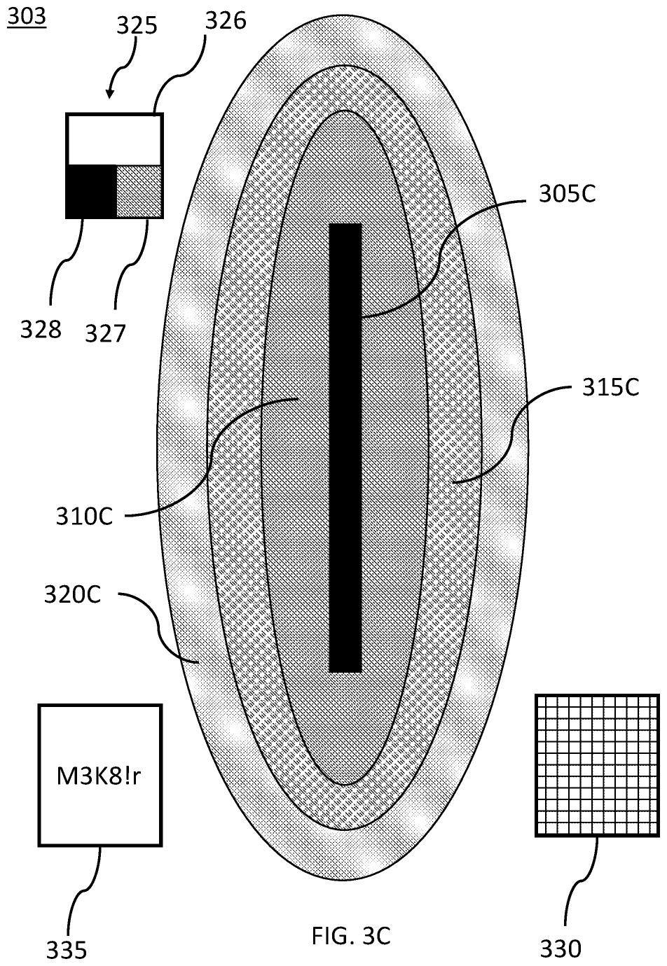

FIGS. 3A-3C provide exemplary illustrations 301, 302, 303, respectively, of images of a wound 305 as well as a first portion of wound surrounding tissue 310, a second portion of wound surrounding tissue 315, and a third portion of wound surrounding tissue 320, wherein the first, second, and third portions of wound surrounding tissue 310, 315, and 320 radiate outward from wound 305 such that first portion of wound surrounding tissue 310 is the closest to wound 305 and third portion of wound surrounding tissue 320 is furthest away from wound 305. More specifically, FIG. 3A is an illustration of wound 305A in the form of a freshly made surgical incision and first, second, and third portions of wound surrounding tissue 310A, 315A, and 320A, FIG. 3B is an illustration of wound 305B a few days following when wound 305A was made and first, second, and third portions of wound surrounding tissue 310B, 315B, and 320B when wound 305B is healing as expected, and FIG. 3C is an illustration of wound 305C a few days following when wound 305A was made and first, second, and third portions of wound surrounding tissue 310C, 315C, and 320C, wherein wound 305C is not healing as expected and/or is infected.

Illustrations 301, 302, 303 of FIGS. 3A-3C, respectively, also include one or more optional information-conveying objects including a color-balancing object 325, a sizing/scaling object 330, and an identifying object 335. Optional information-conveying objects may be, for example, cards or stickers placed on, or near, the wearer's skin proximate to a wound and/or on a wound dressing so that information that may be used to identify and/or assess wound health and/or healing rates that may be captured in an image of the wound. Although a color-balancing object 325, a sizing/scaling object 330, and an identifying object 335 are depicted in FIGS. 3A, 3B, and 3C as three different information-conveying objects, this need not always be the case. For example, patient identifying information, sizing information, and/or color-balancing information may be included in a single object. At times, some, or all, information-conveying objects may be included in and/or a portion of a wound dressing such as augmented wound dressing 101.

Color-balancing object 325 may be of a known color, or group of colors, and may be used by a processor in order to, for example, color correct an image of a wound such as images 301, 302, and/or 303 as, for example, discussed below. In the example of FIGS. 3A-3C, color-balancing object 325 has three portions, each with a different color, wherein a first portion 326 is white, a second portion is a color such as red, and a third portion 328 is black. The color of color-balancing object 325, or portions thereof, may be known via, for example, association of color-balancing object 325 and/or portions of color-balancing object 325 with a known color scheme such as a PANTONE color specifier, which may be either known (e.g., pre-assigned) or provided via on the color-balancing object 325 via, for example, an alpha-numeric and/or optical code.

Sizing object 330 may include information, such as a grid (as shown), an object, and/or a line of a known size that may be used to establish a scale for the wound 305 and surrounding tissue 310, 315, and/or 320 shown in an image and/or illustration. Identifying object 335 may include a code which may be, for example, an alpha-numeric and/or optical code that may be used to identify the wound, the patient, and/or other information such as a date. In some embodiments, identifying object 335 may be received and/or interpreted (i.e., associated with a patient and/or wound) in step 205.

In step 215, sizing and/or color balancing information for the image and/or photograph may be received and/or determined. Sizing information may be, but is not limited to, an object and/or graphic (e.g., cross hairs, a circle, a plus sign, etc.) of known size and/or a measuring device (e.g., ruler) appearing in the image and/or photograph received in step 210 as may be provided by sizing object 330. In some embodiments, the sizing information may be included in a wound dressing. In some cases, this color balancing may be included in the image itself as, for example, an object that has a known color that appears in the image. For example, when a patient is under care for a wound, he or she may be provided with an object (e.g., a white index card or piece of paper) with a known color profile to include in each image of the wound. Additionally, or alternatively, the patient may use an object with a known (or approximately known) color profile such as a piece of white paper or an object that may have a known profile (e.g., a consumer product such as a phone or piece of fabric) for inclusion in an image of the wound. Additionally, or alternatively, the image of the wound may include a digitally-produced color baseline graphic in the form of, for example, an image, color wheel, or graphic that displays on the device (e.g., smart phone, smart watch, or wearable device) that digitally produces the color baseline graphic. In some embodiments, information regarding a camera or other imaging device (e.g., heat or infra-red imaging device) may be received in step 215 and optionally used in step 220 to generate the color-corrected image of the wound.

In some embodiments, the sizing information is received in step 215 via inclusion of a sizing object like sizing object 330 in the image and/or photograph of the wound and the color information may be received in step 215 via inclusion of a color-balancing object like color-balancing object 325 in the image and/or photograph of the wound.

The known color profile of the object in the image of the wound of step 210 may be used as a baseline for understanding the colors included in portions of the image such as the wound itself and surrounding tissue and/or generating a color-corrected image of the wound and surrounding tissue (step 220). Additionally, or alternatively, sizing information for the image (e.g., sizing of the wound may be used to adjust and/or understand a scale and/or size of the wound and/or surrounding portions of tissue.

In step 225, it may be determined whether prior, or historical, information regarding the wound is available. This prior information may include, but is not limited to prior photographs, images, and/or color-corrected images of the wound and/or surrounding tissue. When prior information is available, the color-and/or size-corrected image of step 220 may be compared with the prior information to assess changes therebetween (step 230). In some embodiments, execution of step 230 may also include comparing the color- and/or size-corrected image of step 220 with known wound-healing information such as timelines for proper wound healing, colors present tissue surrounding a wound when the wound is infected, an expected size of a wound following a set number of days since the patient received the wound, etc. In some cases, execution of step 230 may also include comparison of three-dimensional images of a wound to determine a change in texture or degree of swelling associated with the wound.

The comparison of step 230 may, in some cases, involve comparing the images to determine whether the color and/or wound size has changed over time. Such changes include but are not limited to a determination of whether the wound or surrounding tissue has gotten darker or redder (which might indicate infection or cellulitis) or lighter (which may indicate proper healing) in color, and/or has changed color (e.g., has bruising faded or has yellow or colors associated with puss or scabs appeared in the images). The comparison may also look at changes in the shape and/or size of the wound whereby enlarging of the wound may indicate that it is not healing properly.

Optionally, in step 235, additional information about the wound and/or patient may be received. Additional information includes, but is not limited to, whether the patient is immune-compromised and/or may have a slower-than-average healing rate, where the wound is located on the body, medications (e.g., blood thinners, analgesics, etc.) the patient is taking, and/or the type of wound (e.g., incision, cut, tear, burn, etc.).

When prior information is not available and/or following step 235, a determination of a characteristic and/or state of the wound may be made (step 240) and communicated to the user (step 245). Exemplary users included but, are not limited to, the patient, a clinician caring for the patient, and/or a healthcare administrator. Determinations of a characteristic of a wound may include a determination that the wound has changed color or shape, that tissue surrounding the wound has changed color, that cellulitis may have developed proximate to the wound, that puss may have developed on, or proximate to, the wound, and/or a status of scab on a wound. In some embodiments, step 240 may be executed to determine a severity of a problem (e.g., infection, slower-than-average healing rate, etc.) that may be developing within the wound. In some embodiments, execution of step 240 may include analysis of a thermal image of the wound (when, for example, the image of step 210 is a thermal image) to determine a temperature for the wound and/or surrounding tissue to see if the temperature of the wound and/or the surrounding tissue is of an elevated temperature, which may indicate that the wound is becoming infected.

In the example of FIGS. 3A-3C, illustration 301 may be used to establish a baseline shape, size, and/or color for wound 305A, and/or first, second, and/or third portions of wound surrounding tissue 310A, 315A, and 320A. This baseline may be used as, for example, prior wound information in steps 225 and/or 230 and may serve as a basis for the comparison of step 230 for later-taken images/photographs of wound 305. In the example of FIG. 3B, a comparison between image 301 and 302 indicates that a size of wound 305B is smaller than a size of wound 305A and that a color of first, second, and/or third portions of wound surrounding tissue 310B, 315B, and 320B is lighter than a color of first, second, and/or third portions of wound surrounding tissue 310A, 315A, and 320A. These indications of the comparison may indicate that wound 305 is healing as expected in the time between when images 301 and 302 were taken. In contrast, in the example of FIG. 3C, a comparison between image 301 and 303 indicates that a size of wound 305C is larger than a size of wound 305A and that a color of first, second, and/or third portions of wound surrounding tissue 310C, 315C, and 320C is darker than a color of first, second, and/or third portions of wound surrounding tissue 310A, 315A, and 320A. These indications of the comparison may indicate that wound 305 is not healing as expected in the time between when images 301 and 303 were taken and is getting worse and/or could be infected.

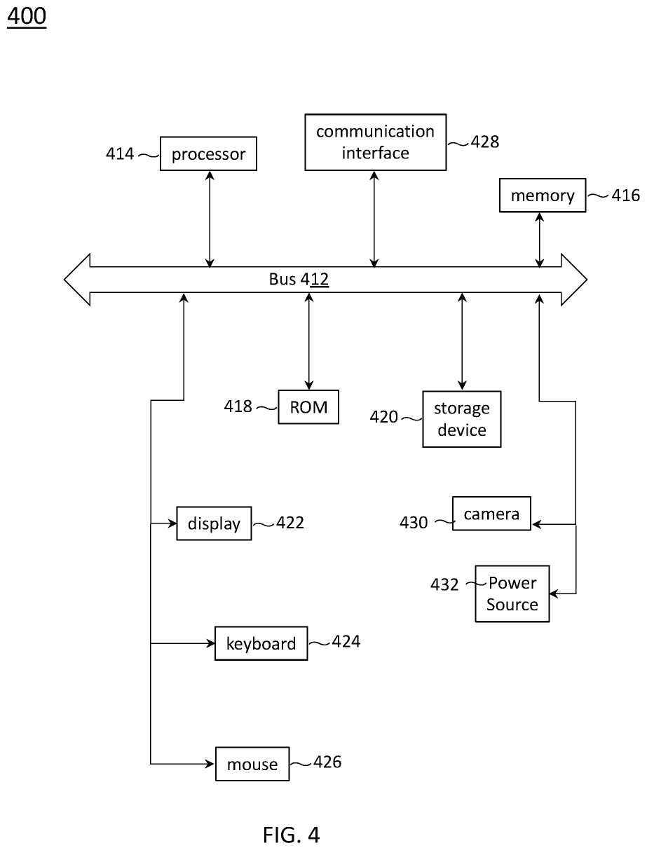

FIG. 4 provides an example of a processor-based system 400 that may store and/or execute instructions for one or more of the processes described herein. Processor-based system 400 may be resident in a server, like server 440 and/or a sanitizing device like sanitizing device 100 and/or 101. Note, not all of the various processor-based systems which may be employed in accordance with embodiments of the present invention have all of the features of system 400. For example, certain processor-based systems may not include a display inasmuch as the display function may be provided by a client computer communicatively coupled to the processor-based system or a display function may be unnecessary. Such details are not critical to the present invention.

System 400 includes a bus 412 or other communication mechanism for communicating information, and a processor 414 coupled with the bus 412 for processing information. System 400 also includes a main memory 416, such as a random-access memory (RAM) or other dynamic storage device, coupled to the bus 412 for storing information and instructions to be executed by processor 414. Main memory 416 also may be used for storing temporary variables or other intermediate information during execution of instructions to be executed by processor 414. System 400 further includes a read only memory (ROM) 418 or other static storage device coupled to the bus 412 for storing static information and instructions for the processor 414. A storage device 420, which may be one or more of a hard disk, flash memory-based storage medium, a magnetic storage medium, an optical storage medium (e.g., a Blu-ray disk, a digital versatile disk (DVD)-ROM), or any other storage medium from which processor 414 can read, is provided and coupled to the bus 412 for storing information and instructions (e.g., operating systems, applications programs and the like).

System 400 may be coupled via the bus 412 to a display 422, such as a flat panel display, for displaying information to a user. An input device 424, such as a keyboard including alphanumeric and other keys, may be coupled to the bus 412 for communicating information and command selections to the processor 414. Another type of user input device is cursor control device 426, such as a mouse, a trackball, or cursor direction keys for communicating direction information and command selections to processor 414 and for controlling cursor movement on the display 422. Other user interface devices, such as microphones, speakers, etc. are not shown in detail but may be involved with the receipt of user input and/or presentation of output.

The processes referred to herein may be implemented by processor 414 executing appropriate sequences of processor-readable instructions stored in main memory 416. Such instructions may be read into main memory 416 from another processor-readable medium, such as storage device 420, and execution of the sequences of instructions contained in the main memory 416 causes the processor 414 to perform the associated actions. In alternative embodiments, hard-wired circuitry or firmware-controlled processing units (e.g., field programmable gate arrays) may be used in place of or in combination with processor 414 and its associated computer software instructions to implement the invention. The processor-readable instructions may be rendered in any computer language.

System 400 may also include a communication interface 428 coupled to the bus 412. Communication interface 428 may provide a two-way data communication channel with a computer network, which provides connectivity to the plasma processing systems discussed above. For example, communication interface 428 may be a local area network (LAN) card to provide a data communication connection to a compatible LAN, which itself is communicatively coupled to other computer systems. The precise details of such communication paths are not critical to the present invention. What is important is that system 400 can send and receive messages and data through the communication interface 428 and in that way communicate with other controllers, etc.

System 400 may also include one or more cameras 430 configured to take one or more images of a wound. In some cases camera 430 may be a three-dimensional camera. In some embodiments, system 400 may further include one or more power sources 432 that may be configured to provide electrical power to system 400.

I claim:

1. A wound dressing comprising:
a wound dressing pad configured to cover a patient's wound; and
a monitor configured to measure an aspect of the patient's health.

2. The wound dressing of claim 1, further comprising:
a wound pad adhesive configured to adhere the wound dressing to the patient and cover the wound.

3. The wound dressing of claim 1 or 2, wherein the wound dressing pad is configured to be elastic.

4. The wound dressing of claim 2 or 3, wherein the wound pad adhesive is configured to be elastic.

5. The wound dressing of any of claims 1-4, wherein the monitor comprises a transmitter configured to communicate a measurement of an aspect of the patient's health to an external computing device.

6. The wound dressing of any of claims 1-5, wherein the monitor is an accelerometer, a humidity monitor, a temperature monitor, one or more electrocardiography leads, a blood oxygen saturation sensor, a tissue oxygen saturation sensor, or a blood sugar sensor.

7. The wound dressing of any of claims 1-6, wherein the monitor is a first monitor, the wound dressing further comprising:
a second monitor affixed to the wound dressing pad, the second monitor being configured to measure another aspect of the patient's health.

8. The wound dressing of claim 7, wherein the second monitor comprises a transmitter configured to communicate a measurement the other aspect of the patient's health to an external computing device.

9. The wound dressing of any of claim 7 or 8, wherein the second monitor is an accelerometer, a humidity monitor, a temperature monitor, one or more electrocardiography leads, a blood oxygen saturation sensor, a tissue oxygen saturation sensor, or a blood sugar sensor.

10. The wound dressing of any of claims 7-9, wherein the first monitor and the second monitor cooperate to monitor the aspect of the patient's health.

11. The wound dressing of any of claims 1-10, wherein the wound dressing pad is configured to absorb moisture.

12. The wound dressing of any of claims 1-11, wherein the wound dressing pad is configured to protect the wound.

13. The wound dressing of any of claims 1-12, wherein the monitor is affixed to the wound dressing pad or the wound dressing adhesive.

14. A method comprising:
receiving, by a processor, identifying information for a wound on a patient;
receiving, by the processor, an image of the wound, the image being associated with color balancing information;
determining, by the processor, a characteristic of the wound using the image and the coloring balancing information; and
communicating, by the processor, the characteristic to a display device for display to a user.

15. The method of claim 14, further comprising:
generating, by the processor, a color-corrected image of the wound; and
communicating, by the processor, the color-corrected image of the wound to the display device for display to the user.

16. The method of claim 14, further comprising:
generating, by the processor, a color-corrected image of the wound, wherein the determining of the characteristic of the wound includes using the color-corrected image of the wound.

17. The method of claim 14, 15, or 16, wherein the image of the wound is a first image of the wound, the color-corrected image of the wound is a first color-corrected image of the wound, and the characteristic of the wound is a first characteristic of the wound, the method further comprising:
receiving, by the processor, a second image of the wound, the second image being associated with color balancing information;
generating, by the processor, a second color-corrected image of the wound;
comparing, by the processor, the first color-corrected image of the wound and the second color-corrected image of the wound;
determining, by the processor, a second characteristic of the wound using a result of the comparing of the first color-corrected image of the wound and the second color-corrected image of the wound; and
communicating, by the processor, the characteristic to a display device for display to a user.

18. The method of any of claims 14-17, wherein the image is a three dimensional image.

19. The method of claim 18, wherein the determined characteristic is a height of the wound or a three dimensional texture of the wound.

20. The method of any of claims 14-19, wherein the determined characteristic is a length of the wound, a width of the wound, a size, or a type of wound.

21. The method of claim 14, wherein the image of the wound is also associated with wound sizing information and the determining the characteristic of the wound uses the wound sizing information to determine a size of the wound.
